(19) United States
(12) Patent Application Publication
(10) Pub. No.: US 2021/0127196 A1
(43) Pub. Date: Apr. 29, 2021

(54) Headphones System and Method

(71) Applicant: Alex Cantwell, Naples, FL (US)

(72) Inventor: Alex Cantwell, Naples, FL (US)

(21) Appl. No.: 17/078,964

(22) Filed: Oct. 23, 2020

Related U.S. Application Data
(60) Provisional application No. 62/925,810, filed on Oct. 25, 2019.

(51) Int. Cl. H04R 1/10; H04R 1/44; A61B 5/0205; A61B 5/00

(52) U.S. Cl. CPC H04R 1/1058; H04R 1/1008; H04R 1/105; H04R 1/44; A61B 5/02438; A61B 5/0205; A61B 5/4875; A61B 5/6803; H04R 2420/07; H04R 1/1025

(57) ABSTRACT

A headband including a headphones system including a battery and electronics and control compartment; and a pair of speakers electrically coupled to the battery and electronics and control compartment; wherein the battery and electronics and control compartment is stitched to fabric of the headband; and wherein the headband including the headphones system is machine washable.

### RELATED APPLICATIONS

This application relates and claims priority to pending U.S. provisional patent application Ser. No. 62/925,810 entitled “Headphones System and Method,” filed on Oct. 25, 2019, by Alex Cantwell.

### FIELD OF THE DISCLOSURE

The invention relates generally to a headphones system and method, and more particularly to a headphones system included within a headband, wherein the headband and headphones system is washing machine washable.

### BACKGROUND

Numerous systems and methods exist for providing music, podcasts, and other performances for individual listening. For the sake of convenience only, and without limitation, reference is made herein only to providing music for individual listening, although the invention described herein is not so limited.

Often, headphones systems and methods are used so that music is provided directly to a person's ears. For example, individuals may wear over-the-ears headphones that are connected to a music player by a cable. While some advancements have been made that eliminate the use of cables or other physical links between headphones and a music player (e.g., through the use of WiFi, Bluetooth, or other connection systems), such systems and methods still are not without their drawbacks.

Many headphones systems are not waterproof. Operation of such devices often stops when the headphones system is placed in or otherwise exposed to water. Accordingly, there is no ability to place such headphones systems in an article of clothing that will be laundered, as the act of washing the item ruins the headphones system. Thus, there remains a need for a simple and cost-effective system and method to provide in a washable clothing item a headphones system.

### SUMMARY

The present disclosure provides a headband including a headphones system that is machine washable, i.e., the headphones system (and each component thereof) is waterproof for up to about thirty minutes when immersed in water up to about one meter deep. In that way, the headband including the headphones may be laundered as desired in conventional washing machines.

Other benefits and advantages of the present disclosure will be appreciated from the following detailed description.

### DESCRIPTION OF THE DRAWINGS

FIG. 1 is a top plan view in partial schematic form of an exemplary embodiment of a headphones system.

FIG. 2 is a bottom plan view in partial schematic form of the exemplary embodiment of a headphones system shown in FIG. 1.

FIG. 3 is a top perspective view in partial schematic form of the exemplary embodiment of a headphones system shown in FIG. 1.

FIG. 4 is a top plan view in partial schematic form of another exemplary embodiment of a headphones system.

FIG. 5 is a top plan view in partial schematic form of the exemplary embodiment of a headphones system shown in FIG. 4, including electrolyte tracking means.

FIG. 6 is a top plan view in partial schematic form of an exemplary embodiment of the headphones system shown in FIG. 4, including heart rate tracking means.

FIG. 7 is a top plan view in partial schematic form of an exemplary embodiment of the headphones system in accordance with the present disclosure.

FIG. 8 is a top plan view in partial schematic form of another exemplary embodiment of the headphones system in accordance with the present disclosure.

FIG. 9 is a side cross-sectional view of another exemplary embodiment of a headphones system in accordance with the present disclosure.

### DETAILED DESCRIPTION

Embodiments of the invention and various alternatives are described. Those skilled in the art will recognize, given the teachings herein, that numerous alternatives and equivalents exist which do not depart from the invention. It is therefore intended that the invention not be limited by the description set forth herein or below.

One or more specific embodiments of the system and method will be described below. These described embodiments are only exemplary of the present disclosure. Additionally, in an effort to provide a concise description of these exemplary embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Further, for clarity and convenience only, and without limitation, the disclosure (including the drawings) sets forth exemplary representations of only certain aspects of events and/or circumstances related to this disclosure. Those skilled in the art will recognize, given the teachings herein, additional such aspects, events and/or circumstances related to this disclosure, e.g., additional elements of the devices described; events occurring related to headphones use; etc. Such aspects related to this disclosure do not depart from the invention, and it is therefore intended that the invention not be limited by the certain aspects set forth of the events and circumstances related to this disclosure.

Turning now to the drawings, the figures show an exemplary headphones system. The system may be embodied in a washable article of clothing, e.g., a headband. The headphones system is machine washable in that it remains waterproof when submerged in up to about one meter of water for up to about thirty minutes.

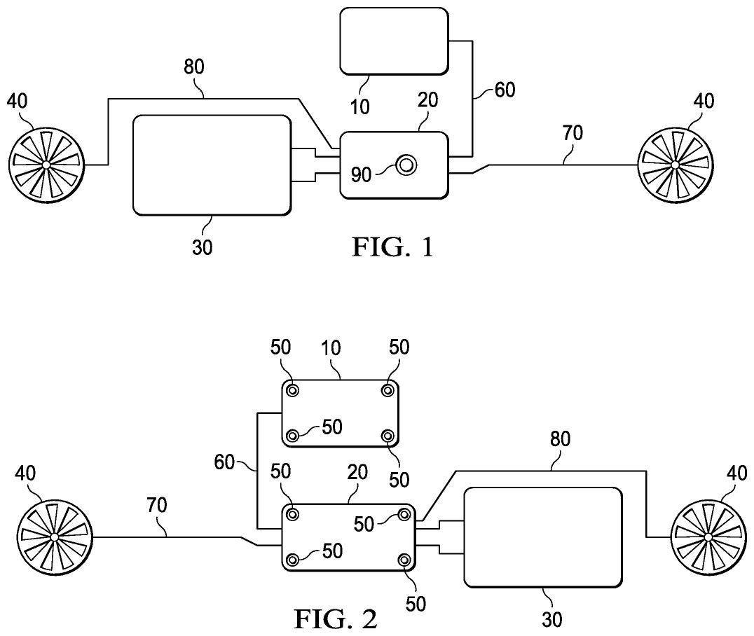

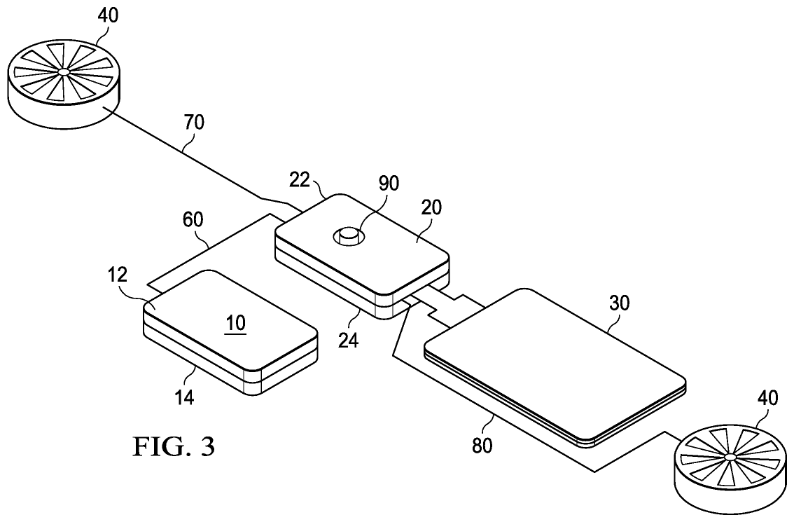

An exemplary embodiment of the headphones system is shown in FIGS. 1, 2 and 3. As described, the headphones module includes four sections: a battery compartment 10; an electronics and control compartment 20; a charging antenna 30; and a pair of speakers 40. As shown in the drawings, the four sections are electrically coupled for operation of the module.

The battery compartment 10 houses a lithium ion battery capable of running the module for up to about six hours. In one embodiment, a 250 mAH battery is used. The form factor of the battery is such that the battery fits within a six-millimeter-thick housing. The housing includes two generally rectangular sections—an upper section 12 and a lower section 14. The sections may include a mating tongue and groove arrangement about their peripheries. An o-ring may be included for additional waterproofing. The sections may be held together by a screw 50 disposed in each corner of the housing. In an alternate embodiment, the sections may be joined by ultrasonic welding.

A power wire 60 electrically couples the battery compartment 10 and the electronics and control compartment 20. The areas where the power wire 60 meets the battery compartment 10 and electronics and control compartment 20 may be sealed with silicone or another such suitable waterproofing material.

The electronics and control compartment 20 may include an upper housing 22 and a lower housing 24. The housings may include mating tongue and groove portions about their peripheries. An o-ring may be held between the housings to help provide waterproofing. The housings may be held together by screws 50. Alternately, ultrasonic welding my be used to join the housings.

The electronics and control compartment 20 is electrically coupled to speakers 40 by speaker wires 70, 80. The compartment 20 is also electrically coupled to charging antenna 30. The charging antenna 30 may be of the type generally commercially available as part of an inductive charging kit. Again, to promote waterproofing each connection point may be sealed with silicone or with another suitable waterproofing material.

The electronics and control compartment 20 houses the electronics for Bluetooth connectivity, for a battery charging circuit, and for a speakers control circuit. The electronics may be designed into a control board that may be disposed in a six-millimeter-thick housing. Electrically coupled to the control board is a button 90. The button 90 on the compartment 20 provides power control. The button 90 also activates syncing via Bluetooth between the headphones system and a music device.

To operate the headphones system, the button 90 is depressed once. A chime sounds through the speakers 40 and power is provided to the system electronics. The headphones system automatically enters a discoverable mode so that the system may be paired via Bluetooth with an audio device. A user selects on the audio device to connect the audio device to the headphones system. A chime may sound through the speakers to indicate a successful connection. Once a connection is made (i.e., once the devices are synced) any audio played on the audio device will be transmitted to the headphones system and heard via the speakers 40. Control of music is accomplished on the audio device (e.g., pause, play, rewind, fast forward, song selection, etc.). Then, to turn off the headphones system the button 90 is pressed and held. The system powers off and a chime may be heard to indicate shutdown.

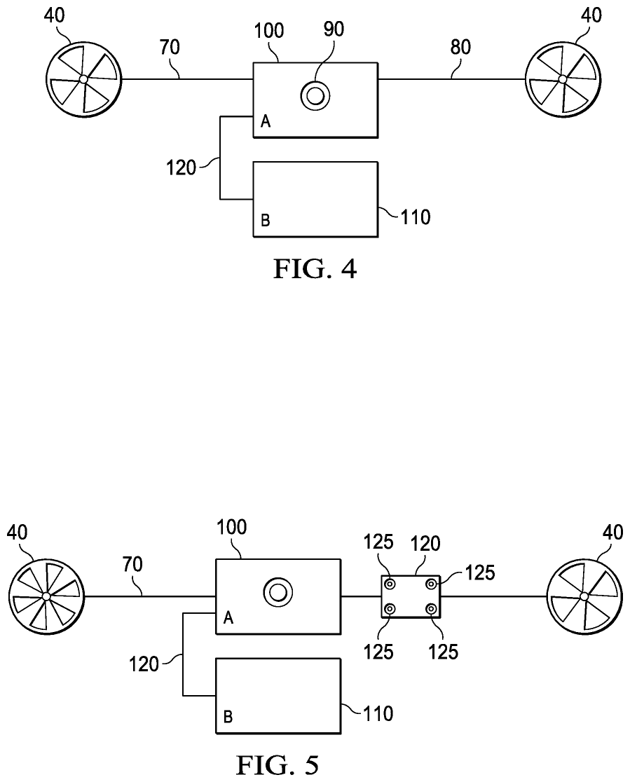

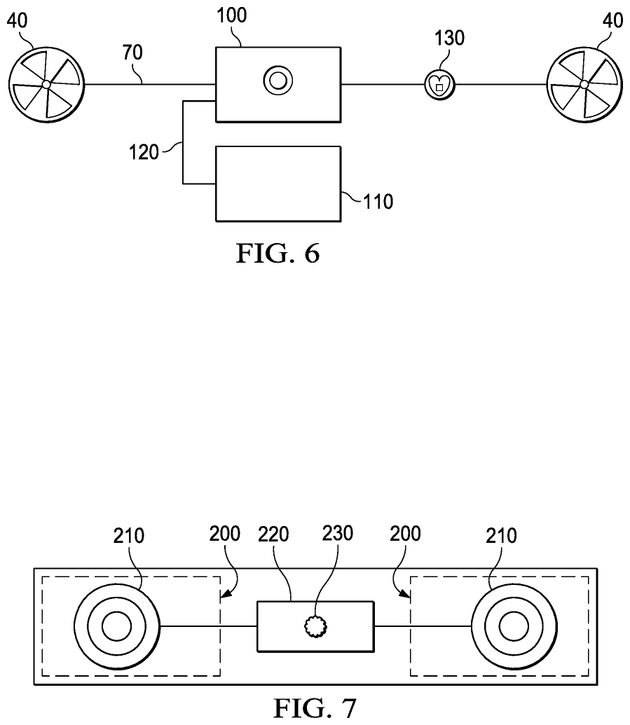

Another embodiment of the invention is shown in FIG. 4. As described, an electronics control compartment 100 is electrically coupled to speakers 40 by speaker wires 70, 80. The compartment 100 is also coupled to battery compartment 110 via line 120. The compartment 100 houses the electronics for Bluetooth connectivity, for a battery charging circuit, and for a speakers control circuit. Again, the electronics may be designed into a control or circuit board electrically coupled to a button 90 to provide power control. The compartment 110 includes a wireless receiver/antenna for charging a battery that is also enclosed in compartment 110. This embodiment provides a simpler waterproofing approach than the previously described embodiment shown in FIGS. 1-3. Each waterproof compartment 100, 110 measures about 51 mm long by 31 mm wide.

Another embodiment is shown in FIG. 5. This embodiment includes an electrode sensor 120 electrically coupled to the control board of compartment 100. The sensor 120 monitors electrolyte levels in a user's sweat. The sensor 120 syncs directly via Bluetooth with an iOS health application. The sensor 120 uses electrodes 125 in contact with the skin to measure electrical conductivity, which data is used to determine real-time electrolyte levels in the user's sweat. Electrolyte level is an indicator of a user's hydration level. In one embodiment, the system communicates hydration status (e.g., hydrated, dehydrated) via a display, via an audible signal, or both. Real-time electrolyte levels, as well as key performance electrolyte levels (high/low) also may be displayed on a chart layout of the synced Bluetooth device display. Also, signals (visual and/or audible) may be provided to the user to indicate that additional electrolyte intake is required to avoid further dehydration.

In another embodiment, the headphones system includes a heart rate sensor 130. The sensor 130 may sync directly via Bluetooth to a commercially available health application to provide real-time heart rate while wearing the headphones system. The sensor 130 may be positioned in the temple area of the head, and provide real-time pulse rate data via Bluetooth to the health app for processing and display.

In accordance with the present disclosure, the headband may be a flexible fabric. In one embodiment, the headband may include one or more of spandex and polyester. More specifically, in one embodiment the headband may include twelve percent spandex and eighty-eight percent polyester fabric.

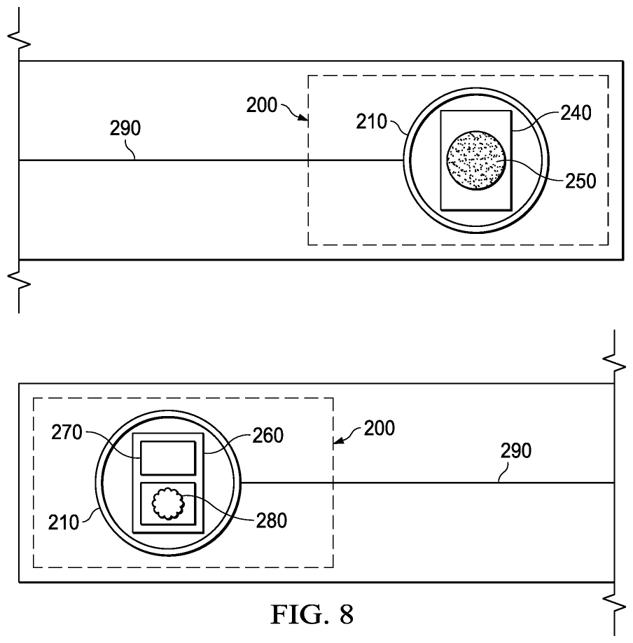

The headband may be formed with one or more compartments 200. See FIGS. 7 and 8. The compartments 200 may comprise a defined volume within which one or more components may be disposed. As shown in FIGS. 7 and 8, speakers/drivers 210 are disposed in compartments 200, so that the speakers/drivers 210 may be adjusted to a desired position in the compartments 200 along the length of the headband.

In one embodiment, shown in FIG. 7, electronic components integrated within the headband include two audio drivers/speakers 210 in two enclosures 200 positioned generally opposite one another, so that the speaker/drivers 210 are fully adjustable to slide over the user's ears dependent on the ear location. A compartment 220 between the two headphones includes a lithium ion battery, a wireless (inductive) charging receiver, a printed circuit board, a Bluetooth chip, and an on/off button 230. The components of compartment 220 are electrically coupled to the speaker/drivers 210.

In another embodiment, shown in FIG. 8, the electronic components integrated within the headband include two speaker/drivers 210 disposed in two generally opposed pockets 200 within the headband. On top of one audio speaker/driver 210 is stacked a lithium ion battery and a wireless (inductive) charging receiver. Stacked on top of one audio driver (left or right) is a lithium ion battery 240, and wireless (inductive) charging receiver 250. Stacked on top of the opposite audio speaker/driver 210 (left or right) is a printed circuit board 260, a Bluetooth chip 270, and an on/off button 280. The two audio speaker/drivers 210 may be electrically coupled by one or more wires 290 positioned along the back of the headband.

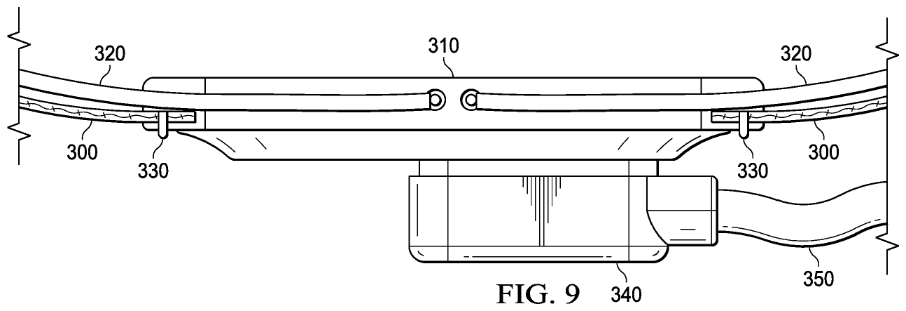

Another exemplary embodiment in accordance with the disclosure is shown in FIG. 9. Fabric 300 of the headband is coupled directly to housing 310 via stitching 330. The housing 310 is electrically coupled via wires 320 to left and right audio drivers (speakers) housed in two separate waterproof enclosures.

The housing 310 may include the following components: one lithium ion battery; one PC board with a power button; a Bluetooth 5.0 chip; a series of LEDs serving as battery level indicators; and an electrode sensor for hydration monitoring by measuring data in sweat, which will sync directly via Bluetooth to an iOS application. The electrode sensor may use electrodes that are in contact with the skin at all times to measure electrical conductivity. Electrical conductivity determines real-time electrolyte levels in sweat, a key indicator of a person's hydration level. Electrolyte levels indicate dehydration or over-hydration.

In addition to the two audio drivers, and waterproof enclosure 310 outlined above, an exemplary embodiment may include a pulse rate sensor embedded into the headband fabric at the temple. This sensor may sync directly via Bluetooth to an iOS application.

The housing 310 may include in one embodiment an inductive charging receiver. Alternately, as shown in FIG. 9, a POGO Pin charging system including a coupler 340 and power cord 350 may be provided.

All of the components may be fully waterproofed to allow the headband to be washed. The housing 310 may be over-molded in silicon, with an extended exterior lip/channel to allow the housing 310 to be stitched into place within the headband with stitches 330 without damaging the compartment or causing potential leaks. A stitched coupling between the housing 310 and headband fabric 300 may allow the enclosure to generally remain in place, while exposing controls and LED's on one side (facing away from the head), and on the opposite side exposing health monitoring sensors (facing towards the head).

This exemplary embodiment will work and operate similarly to the other embodiments disclosed herein, with the addition of a sweat sensor electrolyte monitoring system integrated in parallel with the audio drivers, lithium battery, and PC Board/Bluetooth unit. When the system is turned on with the button on the PC Board, a sound will chime and battery level will be announced. The lithium ion battery will then send power to the audio drivers, Bluetooth Unit, and sensors. The Bluetooth unit may be picked up by any Bluetooth capable device, allowing the headphones system to be synced. When selecting the system from discoverable devices on an iPhone or similar device, the headband may sync automatically; once synced another sound will chime to indicate the system is successfully synced and announce the successful sync (e.g., “Connected to X device”). Once synced music may be played via Bluetooth to the audio drivers. Once synced, data may be collected from the sweat sensor and pulse rate sensor to detect real time electrolyte levels in the sweat that it is reading, as well as heart rate. This data may then be transferred via Bluetooth to an iOS application which may show the user her real time electrolyte levels, as well as key performance electrolyte levels (a high and a low), on a chart layout. The user may also be notified when it would be ideal to intake electrolyte levels to avoid further dehydration.

The headphones system of the present disclosure in one embodiment may be voice controlled and voice activated. Accordingly, the headphones system of the present disclosure may include a microphone electrically coupled to the printed circuit board control circuitry. In one embodiment, the microphone is disposed proximate a speaker/driver. In another embodiment, the microphone is disposed apart from the speakers/drivers.

It should be understood that the foregoing description is only illustrative of the invention. Various alternatives and modifications can be devised by those skilled in the art having the benefit of this disclosure, without departing from the invention. Accordingly, the invention is intended to embrace all such alternatives, modifications and variances.

Certain exemplary embodiments of the disclosure may be described. Of course, the embodiments may be modified in form and content, and are not exhaustive, i.e., additional aspects of the disclosure, as well as additional embodiments, will be understood and may be set forth in view of the description herein. Further, while the invention may be susceptible to various modifications and alternative forms, specific embodiments have been shown by way of example in the drawings and will be described in detail herein. However, it should be understood that the invention is not intended to be limited to the particular forms disclosed. Rather, the invention is to cover all modifications, equivalents and alternatives falling within the spirit and scope of the invention.

What is claimed is:

1. A headband including a headphones system including: a battery and electronics and control compartment; and a pair of speakers electrically coupled to the battery and electronics and control compartment; wherein the battery and electronics and control compartment is stitched to fabric of the headband; and wherein the headband including the headphones system is machine washable.

2. The headband of claim 1, wherein the headphones system is waterproof when submerged up to about one meter deep for up to about thirty minutes.

3. The headband of claim 1, wherein the battery and electronics and control compartment includes a hydration monitoring electrode sensor.

4. The headband of claim 3, including a heart rate sensor electrically coupled to the battery and electronics and control compartment.

5. The headband of claim 1, wherein the electronics and control compartment is overmolded with silicon.

6. A headband including a headphones system including: a battery and electronics and control compartment including a hydration monitoring electrode; and a pair of speakers electrically coupled to the battery and electronics and control compartment.

7. A headband including a headphones system including: a battery and electronics and control compartment; a pair of speakers electrically coupled to the battery and electronics and control compartment; and a heart rate sensor electrically coupled to the battery and electronics and control compartment.
